(19) United States
(12) Patent Application Publication
(10) Pub. No.: US 2008/0294149 A1
(43) Pub. Date: Nov. 27, 2008

(54) Corneal Viewing Chamber

(76) Inventor: Arthur Krolman, Boston, MA (US)

Correspondence Address: BOHAN MATHERS, PO BOX 17707, PORTLAND, ME, 04112-8707, US

(21) Appl. No.: 11/753,985

(22) Filed: May 25, 2007

(51) Int. Cl. A61B 17/00

(52) U.S. Cl. 606/1

(57) ABSTRACT

Viewing chamber for preserving a corneal tissue. The cornea viewing chamber has a container and a lid. The container includes a corneal basket formed by a plurality of prongs for supporting the corneal tissue. The prongs are dimensioned such, that they accommodate corneas ranging in size from large to small. The lid has raised protrusions on its underside, which prevent the corneal tissue from being suctioned onto the lid. The protrusions also serve as a dimension gauge that indicates the size of the tissue stored in the container. Lid and container are threaded. A tapered plug in the lid, as well as the use of an O-ring, enhances the seal when lid is screwed onto the container, ensuring a leak-tight seal. The threaded connection has a thread-stop, to prevent over-torquing of the threaded connection.

### BACKGROUND INFORMATION

1. Field of the Invention

The invention relates to the field of biological specimen containers. More particularly, this invention relates to the field of specimen containers for allograft tissue. More particularly yet, this invention relates to viewing chambers that allow microscopic viewing of the tissue.

2. Description of the Prior Art

The cornea is a transparent component of the eye that covers the iris and pupil. It is responsible for most of an eye's optical power, and helps the eye to focus on an object. Because of its transparent nature, the cornea does not have its own blood supply; instead, it receives nutrients from the tear fluid, the aqueous humour, and from neurotrophins. For these reasons, the cornea is a rather delicate tissue, easily prone to damage and disease. Irreversible, serious vision problems resulting from damage to the cornea are frequently cured today with a corneal transplant.

Before a cornea is transplanted into a recipient's eye, it is sealed in a container that contains a preservation solution, to ensure a sterile environment for the cornea and prevent the introduction of any foreign microorganisms. The donor cornea has a convex side, which is covered with several layers of epithelial cells, which will eventually be entirely replaced by the recipient's own epithelial cells, and a concave side, which is covered with a single layer of irreplaceable endothelial cells. It is important, that the preservation solution flow freely over both sides of the cornea, and particularly, over the endothelial cells.

After excising a cornea from a non-living donor, an eye bank technician typically inspects the donor cornea, to check for any evidence that the donor cornea is unsuitable for transplant. Contraindications for a suitable donor cornea include evidence of: bacterial contamination like conjunctivitis, congenital or acquired disorders like Fuchs dystrophy or a scar, malignant tumors, penetrated foreign bodies like tiny metal filings and refractive surgery like radial keratotomy, lamellar inserts, LASIK, PRK and LASEK.

Conventional corneal transplant preparation requires that the eye bank technician view the corneal tissue with two different types of microscopes. It is, understandably, not desirable to remove the cornea from the storage container for this inspection, because of the risks of exposing the tissue to a non-sterile environment. Thus, the specimen container used to hold the corneal tissue is typically constructed to facilitate such inspection right through the container, which is why the container is commonly referred to as a “viewing chamber”. The technician uses a slit-lamp microscope to check for evidence of any of the contraindications mentioned above and then uses a specular microscope to verify that the proportion of living endothelial cells is adequate to ensure a successful transplant. The specular microscope allows the eye bank technician to focus on a portion of the endothelial layer that is only about 0.3 mm×0.3 mm and count the proportion of living endothelial cells. These cells are fixed in number at birth, thus, by extrapolation, one is able to determine whether sufficient living cells for a successful transplant are present.

Endothelial cells are the most important corneal cells, as they are the “pumping” cells of the cornea and regulate the correct thickness of the tissue. Metabolic waste produced by the endothelial cells often accumulates in the form of sediment that covers the cells on the concave side of a cornea, which may obscure specular microscopic viewing of those cells. It is important, therefore, that the viewing chamber be positionable on its side to permit this waste to drop out of the concave basin, in order to allow unobstructed view of the endothelial cells.

Conventional corneal viewing chambers have several disadvantages. Some of them leak, resulting in reduced amount of preservation solution and the possible ingress of contaminating microorganisms. Given that the corneal viewing chamber is typically tipped on its side during inspection, leakage is a serious problem. Often times, the corneal tissue is suctioned to the lid of the viewing chamber, which causes difficulties when it is time to remove the tissue for transplant. For example, the surgeon may be unable to locate the corneal tissue upon opening the viewing chamber, because it is suctioned up against the underside of the lid. The tissue then often drops to the floor, before the surgeon realizes that it is stuck to the lid, in which case the tissue becomes useless. A corneoscleral disc, suctioned or nearly suctioned against the lid, also prevents the preservation media from flowing freely over the crucial endothelial cells. As a result, any preservation action that the 20 ml volume of media would normally provide is limited to the stagnant tiny volume of fluid trapped between the endothelial cells and the lid.

What is needed, therefore, is a viewing chamber that cradles the corneal transplant tissue, so as to promote flow of the preservation solution over both sides of the tissue. What is further needed is such a chamber that prevents the corneal transplant tissue from being suctioned to the lid. What is yet further needed is such a chamber that is reliably leak proof when placed on its side.

### BRIEF SUMMARY OF THE INVENTION

The present invention is a corneal viewing chamber for preserving corneal tissue or a corneoscleral disc, in preparation of a corneal transplant. The corneal viewing chamber allows clear microscopic viewing of both sides of the corneal tissue, while safely cradling the tissue, so as to protect the delicate corneal cells and promote the flow of preservation solution over both sides of the tissue. The viewing chamber according to the invention eliminates the necessity of removing the corneal tissue from the viewing chamber for inspection prior to transplantation.

The corneal viewing chamber according to the invention has a container that includes a corneal basket for cradling the cornea, and a lid that is screwed onto the container to ensure a sterile environment. The container is filled with conventional preservation fluid, such as, for example, OPTISOL GS from Bausch & Lomb. The corneal basket comprises a plurality of prongs spaced equidistant from each other in a circular array designed to cradle corneal transplant tissue. The prongs have a sloping surface to hold the tissue and are dimensioned and arranged in an array that safely supports corneoscleral discs of various sizes.

External threads are provided around the opening of the container wall and mating internal threads on the lid. A thread-stop is provided at the end of the threaded portion on the container, and the end of the thread on the lid is squared off. The lid is screwed onto the container until the squared-off end hits up against the thread stop. Quite often, in an effort to prevent leakage, an eyebank technician will over-tighten the lid, making it difficult for the next person to open the viewing chamber to retrieve the tissue for transplant. This thread stop prevents over-torquing of the lid and also gives the person securing the lid to the container some feedback, that the lid has been properly screwed onto the container.

The viewing chamber according to the invention provides a dual seal against leakage, in addition to the thread seal: a tapered engagement seal and an O-ring seal. The interior surface of the container wall has a slight V taper. The lid has a male plug arranged concentrically on the inside of the lid. The wall of the plug has a correspondingly slight inverse-V taper. This tapered fit of the lid plug against the tapered interior surface of the container wall forms a leak-proof seal, which maintains its integrity through temperature variations. The lid also has a cavity for receiving an O-ring, which further enhances the leak-tight seal. The combination of the taper engagement seal and the O-ring provides an effective leak-proof seal to maintain a sterile environment and prevent contamination of the corneal tissue by foreign microorganisms.

The viewing chamber according to the invention is constructed to prevent the corneal transplant tissue from being suctioned up against the underside of the lid. The central portion of the lid has a viewing window, which enables examination of the endothelial cells on the corneal tissue. Protrusions are provided on the underside of the lid, in a circular arrangement around the perimeter of the endothelial viewing window. The protrusions are shapes or forms that are raised from the plane of the underside of the lid. If the viewing chamber is inverted and the corneal transplant tissue settles onto the underside of the lid, the protrusions create a gap between the tissue and the lid at a plurality of locations around the perimeter of the tissue, thereby ensuring that preservation fluid flows between the tissue and the lid, and thereby preventing the corneal tissue from suctioning up against the underside of the lid.

### BRIEF DESCRIPTION OF THE DRAWINGS

The present invention is described with reference to the accompanying drawings. In the drawings, like reference numbers indicate identical or functionally similar elements.

FIG. 1 shows a side view of the corneal viewing chamber according to the invention.

FIG. 2 is a perspective view on an inner portion of the viewing chamber of FIG. 1, illustrating the array of prongs that form the corneal basket.

FIG. 3 is a side view of the first and second prongs, illustrating the dimensional differences,

FIG. 4 is a bottom plane view of the lid, illustrating the dimension gauge and anti-suction protrusions on the underside of the lid.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

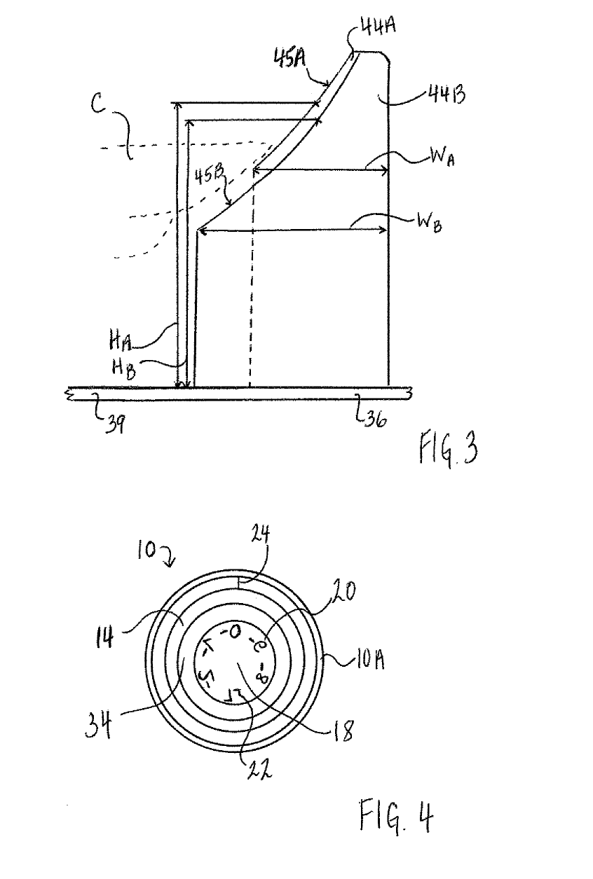

FIG. 1 is a cross-sectional view of a corneal viewing chamber 100 according to the invention, for viewing and preserving a corneoscleral disc C, which is partially shown in dashed lines in FIG. 3, and which is also referred to hereinafter as corneal tissue. The corneal viewing chamber 100 comprises essentially a container 30 and a lid 10, which, when screwed together, form a leak-tight container. A corneal basket 40, to be discussed below, is provided inside the container 30. A threaded opening 32 is provided at the top of the container 30. In the embodiment shown, the container 30 has an inner wall 31, an outer wall 33, and a container base 36 that includes a first viewing window 39 for viewing epithelial cells. The inner wall 31 and outer wall 33 are connected to each other by several connector walls 35, that are spaced radially about the container 30. The first viewing window 39 is recessed into the base 36 such that, when the container 30 is placed in its upright position on a plane surface, the container is supported by the outer wall 33; the base 36 and the viewing window 39 are raised above the plane surface sufficient to prevent any scratches to the window, yet close enough to the lower plane of the base 36, so as not to impede wide-angle viewing with a slit-lamp microscope. The inner wall 31 has a slight V-shaped taper 31A. The threaded opening 32 is provided with external threads 32A and a thread stop 32B.

The lid 10 has an outer wall 12 with threads 12A, and a second viewing window 18 for viewing endothelial cells. When the lid 10 is screwed onto the container 30, the first viewing window 39 is aligned directly beneath the second viewing window 18. Arranged concentrically in the center of the lid 10 is a plug 14, which has a second taper 14A in an inverted V-shape, which is dimensioned to sealingly engage with the first taper 31 on the container 30. Engaging the threads 12A on the lid 10 with the threads 32A on the container 30 forces the plug 12 into close contact with the first taper 31A of the inner wall 31. The lid 10 also has a cavity 16 for receiving an O-ring 15. The O-ring 15 may be made of silicone or other suitable sealant material. Together, the meshed threads 12A and 32A, the close fit of the first taper 31A and the second taper 14A, and the O-ring seal 15 provide a leak-proof seal that prevents the ingress of foreign or contaminating microorganisms into the sterile environment in the sealed viewing chamber 100.

The thread 12A on the lid 10 terminates with a squared-off stop surface 12B. A stop 32B is provided on the threaded opening 32 of the container 30. When the lid 10 is screwed onto the container 30, the stop 32B prevents the screw connection from being over-torqued. This ensures that the lid 10 is readily removable from the container 30 with the application of reasonable disassembly torque.

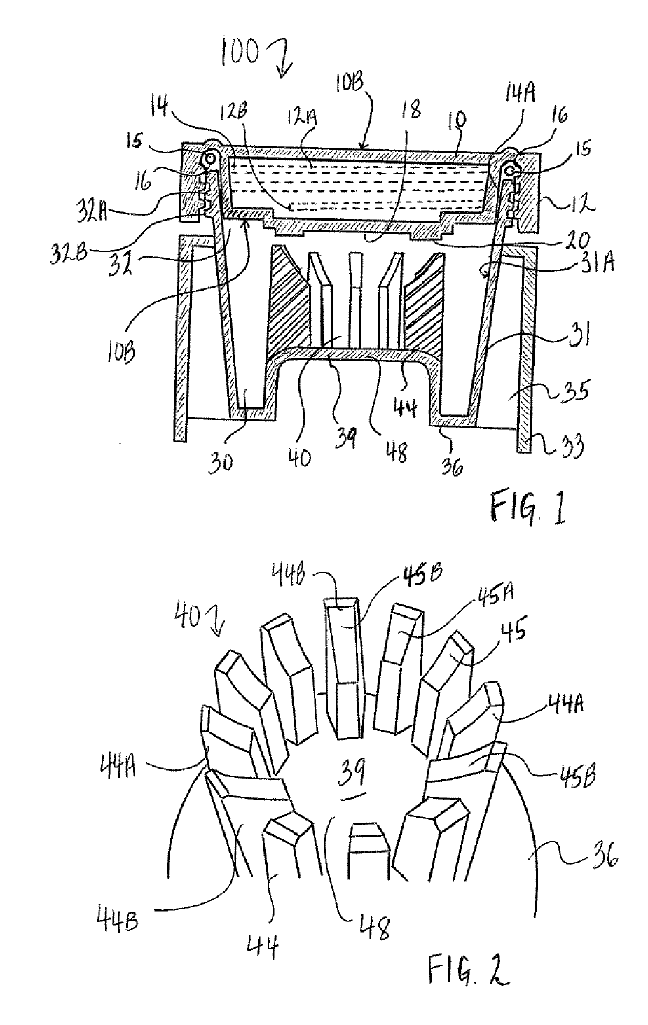

FIGS. 2 and 3 illustrate the corneal basket 40, which is disposed in the center of the container 30 on the base 36, above the first viewing window 39. The corneal basket 40 comprises an array of prongs 44 arranged in a radial formation to support the corneoscleral disc C. The prongs 44 are spaced radially equidistant from each other, which provides space for conventional forceps to be inserted into the basket 40 to grasp and retrieve the corneal tissue during a transplant procedure. Each prong 44 has a sloping support surface 45 shaped to match the curvature of the average human eye globe, which has an average diameter of 25 mm. The array of prongs 44 includes prongs of various dimensions, arranged to properly support corneoscleral discs C of various sizes. In the embodiment shown, the basket 40 includes first prongs 44A and second prongs 44B. FIG. 3 is a close-up detail drawing, showing a dimension comparison between prongs 44A and 44B. The first prong 44A has a first height dimension H_{A} and a first width dimension W_{A}; the second prong 44B has a second height dimension H_{B} and a second width dimension W_{B}. As can be seen, the second width dimension W_{B} is slightly greater than the first width dimension W_{A} and extends farther in toward the center of the basket 40. These drawings are not to scale, but the difference W_{B}−W_{A} is preferably approximately 1.5 mm. The second height dimension H_{B} of the second prong 44B is slightly less than the first height dimension H_{A}. In the embodiment shown, the array of prongs 44 includes nine first prongs 44A and three second prongs 44B, arranged radially as a repeating series of three first prongs 44A and one second prong 44B. This arrangement of first prongs 44A and second prongs 44B provides a secure cradle for corneoscleral discs C ranging in diameter from 17 mm to 22 mm. The corneoscleral disc C is placed in the cradle with the convex side facing downward toward the viewing window 39 in the base 36 of the container. A major concern in storing such corneal tissue is to prevent unnecessary abrasion of the epithelial cells. Ideally, only the outer edge of the corneoscleral disc C comes into contact with the prongs 44, as illustrated in FIG. 3. Although a cornea recipient will grow a new epithelial layer, some transplant surgeons specifically request donor corneas with an intact epithelial layer, because this allows the protective bandages to be removed from a patient earlier than if the patient had received a cornea with a damaged epithelial layer. These surgeons feel that the transplant recipient will have a more positive short-term attitude about the success of the transplant operation, if the bandages are removed earlier. For this reason, it is desirable to minimize abrasion contact of the prongs 44 with the epithelial layer. The second prongs 44B, with their greater width dimension W_{B} and lower height dimension H_{B}, normally do not come into contact with the corneal tissue, as long as the tissue is properly cradled in the basket 40. Thus, even though the sloping support surface 45B extends farther in toward the center of the corneoscleral disc C, the first prongs 44A generally cradle the disc far enough above the sloping support surface 45B, so that the epithelial layer generally does not come into contact with the second prongs 44B. Should the corneal tissue start to tip sideways, however, the second prongs 44B serve to right the tissue back onto the cradle provided by the first prongs 44A and prevent the tissue from falling sideways into the bottom of the corneal basket 40.

FIG. 4 is a bottom plane view of the lid 10, which shows a feature that functions simultaneously as a dimension gauge and as an anti-suction means that prevents the corneoscleral disc C stored in the viewing chamber 100 from being suctioned up against the underside 10A of the lid. Protrusions 20 are provided on the underside 10A. These protrusions 20 are positioned about the perimeter of the second viewing window 18, so as not to obscure inspection of the corneal tissue while it is stored in the chamber 100. The protrusions 20 hold the corneoscleral disc C a small distance away from the surface of the underside 10A of the lid 10, which ensures that preservation solution flows between the tissue and the lid 10. Thus, the endothelial cells remain bathed in the solution at all times, and should the tissue float up against the lid 10, the flow of the solution between the tissue and the lid 10 prevents the disc C from suctioning up against the lid 10. The protrusions 20 may be any shape, just as long as they are raised from the surface of the underside 10A sufficiently to ensure that preservation solution flows between the corneal tissue C and the lid 10.

In the embodiment shown, the protrusions 20 also serve as a dimension gauge. For this purpose, the protrusions 20 include a first series of marks that is a size indicator 21 and second series of marks that is a size gauge 22. The marks of either one or both of the first series and second series may be formed as the protrusions 20, but at least one of the series must be, to ensure the anti-suction effect described above. In this embodiment, the first series, i.e., the size indicator 21, comprises a series of marks that are shaped as the Arabic numerals 7, 8, 9, 0, 1, and 2, although it is understood that other representations may be used. The numerals 7, 8, 9, 0, 1, and 2 indicate the diameters 17 mm, 18 mm, 19 mm, 20 mm, 21 mm and 22 mm, respectively. The second series of marks, i.e. the size gauge 22, comprises a series of lines, each one being a measurement of a specific diameter of a disc centered in the corneal basket 40. Each size indicator mark is paired with a specific size gauge mark. When the corneoscleral disc C is properly supported by the prongs 44, it is concentrically aligned within the basket 40. The outer edge of the disc C touches or come closest to the size gauge mark 22 that corresponds to the actual circumferential dimension of the disc. The eye bank technician is now able, using this useful dimension gauge, to record the size of the corneoscleral disc C simply by viewing it through the second viewing window 18.

The container 130 is made of a clear plastic polymer, such as polymethyl methacrylate (PMMA), or other suitable material used to contain biological specimens or tissue and to allow microscopic viewing of the corneoscleral disc C through the viewing windows 18 and 39.

It is understood that the embodiments described herein are merely illustrative of the present invention. Variations in the construction of the corneal viewing chamber may be contemplated by one skilled in the art without limiting the intended scope of the invention herein disclosed and as defined by the following claims.

What is claimed is:

1. A viewing chamber for storing and viewing corneal tissue, said viewing chamber comprising:
a container having a corneal basket arranged within said container on a container base, said corneal basket adapted to support a corneoscleral disc; and
a lid having a lid underside with protrusions on said lid underside that are formed so as to ensure a gap and thereby a flow of a preservation fluid between said corneoscleral disc and said lid underside and thereby to prevent said corneoscleral disc from suctioning up against said lid underside.

2. The viewing chamber of claim 1, wherein said lid includes a lid viewing window and wherein said protrusions are arranged radially about a perimeter of said lid viewing window.

3. The viewing chamber of claim 1, wherein said protrusions serve as dimension gauge for determining a dimension of said corneoscleral disc supported within said corneal basket.

4. The viewing chamber of claim 3, wherein said dimension gauge comprises a series of said protrusions, each protrusion of said series representing a specific dimension.

5. The viewing chamber of claim 4, wherein said series of protrusions includes a first series of protrusions that is a size indicator, and a second series of protrusions that is a size gauge, each protrusion of said second series being paired with one of said protrusions of said first series, and wherein, when said corneoscleral disc is supported in said corneal basket and an outer perimeter of said corneoscleral disc aligns closest with one of said second series of protrusions, said one of said second series indicates a diameter of said corneoscleral disc and said paired one of said first series indicates a size of said corneoscleral disc.

6. The viewing chamber of claim 1, wherein said corneal basket comprises a plurality of first prongs that are adapted to support said corneoscleral disc, said first prongs having a disc support surface that corresponds in curvature to a natural curvature of said corneoscleral disc.

7. The viewing chamber of claim 6, said corneal basket comprising a plurality of second prongs;
wherein said first prongs have a first prong height of said disc support surface and a first prong width and said second prongs have a second prong height of said disc support surface and a second prong width, and wherein said second prong height is lower than said first prong height and said second prong width is greater than said first prong width, such that said plurality of second prongs provides a support surface that is lower in height and smaller in diameter than a support surface formed by said first prongs.

8. The viewing chamber of claim 7, wherein said plurality of first prongs includes nine prongs and said plurality of second prongs includes three prongs, and wherein said corneal basket is formed by radially arranging repeating sets of three of said first prongs and one of said second prongs on said container base.

9. The viewing chamber of claim 8, wherein said plurality of first prongs is adapted to support said corneoscleral disc above said support surface of said plurality of second prongs.

10. The viewing chamber of claim 1, said container base having a first plane and a second plane that is recessed relative to said first plane, and wherein a container viewing window is provided in said second plane.

11. The viewing chamber of claim 1 further comprising a seal system that includes a threaded seal that includes a container thread on said container and a lid thread on said lid, wherein said container thread has a thread stop and said lid thread has a squared-off end, such that, when said lid is screwed onto said container, said squared-off end is stopped by said thread stop, so as to prevent over-torquing of said threaded seal.

12. The viewing chamber of claim 11, said seal system further comprising an O-ring seal, said lid having a recess for receiving an O-ring that is adapted to sealingly press against an upper edge of said container when said lid is sealingly engaged with said container.

13. The viewing chamber of claim 11, said seal system further comprising a taper seal, said container having an inner container wall and said lid having an inner lid wall, wherein said inner container wall has a first taper and said inner lid wall a second taper, and wherein, when said lid is sealingly engaged with said container, said second taper is forced against said first taper to form a taper seal, so as to prevent flow of fluid past said taper seal into said lid.

14. A viewing chamber for storing and viewing corneal tissue comprising:
a container having a container viewing window in a container base and a corneal basket arranged within said container on said container base, said corneal basket adapted to support a corneoscleral disc; and
a lid that sealingly engages with said container;
wherein said corneal basket includes a plurality of prongs that are adapted to support said corneoscleral disc, said prongs having a sloping disc support surface that corresponds in curvature to a natural curvature of said corneoscleral disc, and wherein said prongs are radially arranged on said container base, about a perimeter of said container viewing window.

15. The viewing chamber of claim 14, wherein said plurality of prongs includes a plurality of first prongs, each first prong of said plurality of first prongs having a first prong height of said disc support surface and a first prong width, and a plurality of second prongs, each second prong of said plurality of second prongs having a second prong height of said disc support surface and a second prong width, and wherein said second prong height is lower than said first prong height and said second prong width is greater than said first prong width, such that said plurality of second prongs provides a support surface that is lower in height and smaller in diameter than a support surface formed by said first prongs.

16. The viewing chamber of claim 15, wherein said plurality of first prongs includes nine prongs and said plurality of second prongs includes three prongs, and wherein said corneal basket is formed by radially arranging on said container base repeating sets of said prongs comprising three of said first prongs and one of said second prongs; and
wherein said plurality of first prongs is adapted to support said corneoscleral tissue above said sloping support surface of said second prongs.

17. The viewing chamber of claim 14, wherein said lid has an underside, said viewing chamber further comprising a combination of an anti-suction means and a dimension gauge on said lid underside, said anti-suction means comprising a series of protrusions that protrude away from a plane of said lid underside and are adapted to prevent said corneoscleral disc from suctioning up against said lid underside by allowing fluid flow between said lid underside and said corneoscleral disc.

18. The viewing chamber of claim of claim 17, wherein said protrusions include a first series of protrusions that is a size indicator and a second series of protrusions that is a size gauge, each protrusion of said second series being paired with one of said protrusions of said first series, and wherein, when said corneoscleral disc is supported in said corneal basket and an outer perimeter of said corneoscleral disc aligns closest with one of said second series of protrusions, said one of said second series indicates a diameter of said corneoscleral disc and said paired one of said first series indicates a size of said corneoscleral disc.

19. The viewing chamber of claim 17, wherein said first series of protrusions indicate millimeter dimensions and said second series measures a diameter within said corneal basket.

20. The viewing chamber of claim 14 further comprising a multi-seal system that includes a threaded seal, said lid and said container having mating threads to sealingly engage each other, and an O-ring seal, said lid having a recess for receiving an O-ring, and a taper seal, said container having an inner wall with a first taper and said lid having an outer wall with a second taper, and wherein, when said lid sealingly engages with said container, said mating threads provide a first seal, said O-ring presses down against an upper edge of said container, so as to provide a second seal, and said second taper is forced against said first taper, so as to form said taper seal.
